(19) United States
(12) Patent Application Publication
(10) Pub. No.: US 2009/0099151 A1
(43) Pub. Date: Apr. 16, 2009

(54) Modified Release Pharmaceutical Compositions and Processes Thereof

(75) Inventors: Rajesh Jain, New Delhi (IN); Kour Chand Jindal, New Delhi (IN); Sukhjeet Singh, New Delhi (IN); Sanjay Boldhane, New Delhi (IN)

Correspondence Address: LADAS & PARRY LLP, 26 WEST 61ST STREET, NEW YORK, NY, 10023, US

(73) Assignee: PANACEA BIOTEC LTD., New Delhi (IN)

(21) Appl. No.: 11/922,961

(22) PCT Filed: Jun. 29, 2006
(86) PCT No.: PCT/IN2006/000224

§ 371 Date: Dec. 27, 2007

(30) Foreign Application Priority Data
Jun. 29, 2005 (IN) 1681/DEL/2005

(51) Int. Cl. A61K 31/554; A61K 31/351; A61K 31/436; A61K 31/404; A61K 31/138; A61K 31/52; A61K 31/55; A61K 31/40; A61K 31/505

(52) U.S. Cl. 514/211.13; 514/460; 514/291; 514/419; 514/423; 514/275; 514/217; 514/654; 514/263.4

(57) ABSTRACT

Novel modified release pharmaceutical composition comprising at least one active agent(s); a polymer system in an amount less than about 80% w/w of the composition comprising at least two swellable pH independent polymers wherein at least one is hydrophilic; optionally other pharmaceutically acceptable excipients is provided. Process for preparation of such compositions and methods of using them is also provided. The compositions are formulated into suitable dosage forms that provide therapeutic concentrations of active agent for extended periods of time.

### FIELD OF THE INVENTION

The present invention relates to novel modified release pharmaceutical composition comprising at least one active agent(s) or its pharmaceutically acceptable salts, esters, prodrugs, solvates, hydrates, or derivatives thereof; a polymer system in an amount less than about 80% w/w of the composition comprising at least two swellable pH independent polymers wherein at least one is hydrophilic; optionally other pharmaceutically acceptable excipients; wherein the composition provides therapeutic concentrations of active agent(s) for extended periods of time. The present invention also describes process for preparation of such compositions and method of using such compositions. The controlled release composition is useful in providing therapeutically effective levels of the said active agent(s) for extended periods of time.

### BACKGROUND OF THE INVENTION

Drug levels can be maintained above the lower level of the therapeutic plasma concentration for longer periods of time by administering larger doses of conventionally formulated dosage forms, but this approach might produce toxic effects due to high plasma concentration of the drug. Alternatively, another approach is to administer a drug at certain intervals of time, resulting in oscillating drug levels, the so-called peak and valley effect. This approach is generally associated with several potential problems, such as a large peak (toxic effect) and valley (non-active drug level) effect, and a lack of patient compliance leading to drug therapy inefficiency or failure. To overcome such issues, modified release compositions can be formulated with the objective of releasing the drug in a sustained or controlled manner for an extended period of time.

A number of drugs collectively known as statins have been known to reduce serum LDL cholesterol levels. High LDL cholesterol levels have been shown to be an important risk factor in the development of arteriosclerosis and ischemic heart disease. Statins have been found to lower serum LDL cholesterol levels in a dose dependent manner. Additionally, these drugs lower serum triglyceride levels, which is another risk factor for heart disease. Statins lower serum LDL cholesterol levels by competitive inhibition of 3-hydroxyl-3-methylglutaryl-Coenzyme A reductase (HMG CoA reductase), an enzyme involved in the biosynthesis of cholesterol. By binding tightly to the active site of the enzyme, statins block the reduction of HMG CoA, a step necessary in the biosynthesis of cholesterol. This inhibition of cholesterol biosynthesis by a statin results in a decrease in the production and secretion of LDL cholesterol. In addition, the upregulation of LDL receptors, especially in the liver, leads to the removal of LDLs from the serum. Thus, by reducing the production of LDL cholesterol and by causing LDL cholesterol to be removed from the serum, statins effectively reduce overall serum LDL cholesterol levels.

Atorvastatin and Simvastatin are cholesterol-lowering agents. Atorvastatin is a selective, competitive inhibitor of HMG-CoA reductase, the rate-limiting enzyme that converts 3-hydroxy-3-methylglutaryl-coenzyme A to mevalonate, a precursor of sterols, including cholesterol. Simvastatin is a pro-drug derivative of lovastatin. After absorption, it undergoes rapid enzymatic hydrolysis of the lactone ring to form the principal metabolite, simvastatin-p-hydroxyacid. This metabolite acts as a potent, reversible, competitive inhibitor of BMG CoA reductase which catalyses the conversion of hydroxymethyl glutarate to mevalonate. This conversion is an early and rate-limiting step in the biosynthesis of cholesterol. Simvastatin inhibits the production of cholesterol by the liver and lowers overall blood cholesterol as well as blood LDL cholesterol levels. Tacrolimus is a macrolide immunosuppressant produced by Streptomyces tsukubaensis. It inhibits T-lymphocyte activation, although the exact mechanism of action is not known. Quetiapine is a psychotropic agent belonging to the chemical class of dibenzothiazepine derivatives. It is an antagonist at multiple neurotransmitter receptors in the brain: serotonin 5HT_{1A} and 5HT₂, dopamine D₁ and D₂, histamine H₁, and adrenergic α₁ and α2 receptors. Oxcarbazepine is an antiepileptic drug, which exerts the pharmacological activity primarily through the 10-monohydroxy metabolite (MHD) of oxcarbazepine. Levetiracetam is an antiepileptic drug indicated as adjunctive therapy in the treatment of partial onset seizures in adults and children 4 years of age and older with epilepsy. Tolterodine is a competitive muscarinic receptor antagonist and is indicated for the treatment of overactive bladder with symptoms of urge urinary incontinence, urgency and frequency. Famciclovir is an orally administered prodrug of the antiviral agent penciclovir. It undergoes rapid biotransformation to the active antiviral compound penciclovir, which has inhibitory activity against herpes simplex virus types 1 (HSY-1) and 2 (HSV-2) and varicella zoster virus (VZV).

In the past, several attempts have been made to develop modified release/controlled release formulations comprising various drugs. Such modified/controlled release compositions provide better patient compliance as compared to conventional compositions since they need to be administered generally only once daily as compared to b.i.d. or t.i.d. dosage form.

U.S. Pat. No. 4,444,784 describes an antihypercholesterolemic agent, which is known as simvastatin, which is an effective HMG-CoA reductase inhibitor. Simvastatin is commercially sold in the US and elsewhere under the brand name ZOCOR® by Merck & Company, Inc. U.S. Pat. No. 5,916,595 describes a controlled release formulation comprising a compressed tablet core which contains an alkyl ester of a hydroxy substituted naphthalene compound, a pharmaceutically acceptable, water swellable polymer and an osmotic agent; and an outer coating layer which completely covers the osmotic core and comprises a pH sensitive coating agent, a channeling agent and a water insoluble cellulosic polymer used at a weight ratio of 0.1:1 to 0.75:1 and at a combined coating weight of 0.5-5% by weight.

U.S. Pat. No. 5,007,790, U.S. Pat. No. 5,582,837 and U.S. Pat. No. 5,972,389 describes sustained release dosage forms for oral administration, designed to deliver a pharmacologically active agent to the stomach and gastrointestinal tract over an extended time period. The dosage forms described in the aforementioned patents are comprised of particles of a hydrophilic, water-swellable polymer with the drug dispersed therein. The polymeric particles in which the drug is dispersed absorb water, causing the particles to swell, which in turn promotes their retention in the stomach and also allows the drug contained in the particles to dissolve and then diffuse out of the particles. The polymeric particles also release drug due to physical erosion or degradation.

The PCT publication nos. WO03035041 and WO03035029 primarily disclose a controlled release oral dosage form for the continuous, sustained administration of a pharmacologically active agent, wherein the composition comprises a matrix of a biocompatible, hydrophilic, erodible polymer with an active agent incorporated therein, wherein the polymer is one that both swells in the presence of water and gradually erodes over a time period of hours, with swelling and erosion commencing upon contact with gastric fluid.

The PCT publication bearing no. WO200421972 describes a formulation comprising a core comprising at least one poorly water-soluble statin and at least one surface-active agent; and a polymeric membrane-controlled coating comprising less than 50% by weight of at least one water-soluble or water-permeable polymer and greater than 50% by weight of at least one water-insoluble or water-impermeable polymer and methods of use of such compositions in treating, preventing, and/or managing one or more cardiovascular diseases.

Another PCT publication bearing no. WO2004002445 provides a novel gastro-retentive delivery system for controlled release of active agent in stomach or upper part of gastrointestinal tract in the form of bilayer dosage form in which one layer (Layer-A) is responsible for retaining the dosage form in stomach or upper part of gastrointestinal tract (spatial control) for prolonged period and is composed of pharmaceutical excipients with low bulk density such as cellulosic derivatives either natural, semi-synthetic or synthetic, ethyl cellulose in particular polyethylene oxide, fatty acids, hydrogenated oils, waxes, shellac, and the likes either alone or in combination and along with other optional pharmaceutical excipients. The second layer (Layer-B) is responsible for prolonged or controlled drug delivery (temporal control) and comprises of controlled release matrix polymers such as synthetic or semisynthetic cellulose derivatives like hydroxypropyl methylcellulose, ethylcellulose and the like and/or natural polymers or gums such as xanthan gum, gelatin, acrylic acid derivatives, polyvinyl acetate and the like along with other optional pharmaceutical excipients.

Several attempts to provide dosage forms for delivery of active agents that remains in the stomach for extended periods of time has been described previously. However, there still exists a need to develop modified release dosage form compositions which can provide sustained delivery of active agent, which are easier to manufacture and involves a low formulation cost. The present invention provides such novel modified release compositions.

### SUMMARY OF THE INVENTION

It is an objective of the present invention to provide novel modified release pharmaceutical composition comprising at least one active agent(s) or its pharmaceutically acceptable salts, esters, prodrugs, solvates, hydrates, or derivatives thereof; a polymer system in an amount less than about 80% w/w of the composition comprising at least two swellable pH independent polymers wherein at least one is hydrophilic; optionally other pharmaceutically acceptable excipients; wherein the composition provides therapeutic concentrations of active agent(s) for extended periods of time.

It is, an objective of the present invention to provide novel modified release pharmaceutical composition comprising at least one active agent(s) or its pharmaceutically acceptable salts, esters, prodrugs, solvates, hydrates, or derivatives thereof; a polymer system in an amount less than about 80% w/w of the composition comprising at least two swellable pH independent polymers in a ratio of 1:20 to 20:1 wherein at least one is hydrophilic; optionally other pharmaceutically acceptable excipients; wherein the composition provides therapeutic concentrations of active agent(s) for extended periods of time.

It is an objective of the present invention to provide novel modified release pharmaceutical composition comprising at least one active agent(s) or its pharmaceutically acceptable salts, esters, prodrugs, solvates, hydrates, or derivatives thereof; at least one diluent(s) in an amount greater than 5% w/w of the composition; a polymer system in an amount less than about 80% w/w of the composition comprising at least two swellable pH independent polymers in a ratio of 1:20 to 20:1 wherein at least one is hydrophilic; optionally other pharmaceutically acceptable excipients; wherein the composition provides therapeutic concentrations of active agent(s) for extended periods of time.

It is an objective of the present invention to provide novel modified release pharmaceutical composition comprising at least one active agent(s) or its pharmaceutically acceptable salts, esters, prodrugs, solvates, hydrates, or derivatives thereof; a polymer system in an amount less than about 80% w/w of the composition comprising at least two swellable pH independent polymers in a ratio of 1:20 to 20:1 wherein at least one is hydrophilic and additionally, at least one pH dependent hydrophilic release controlling polymer; optionally other pharmaceutically acceptable excipients; wherein the composition provides therapeutic concentrations of active agent(s) for extended periods of time.

It is an objective of the present invention to provide novel modified release pharmaceutical composition comprising at least one active agent(s) or its pharmaceutically acceptable salts, esters, prodrugs, solvates, hydrates, or derivatives thereof; at least one diluent(s) in an amount greater than 5% w/w of the composition; a polymer system in an amount less than about 80% w/w of the composition comprising at least two swellable pH independent polymers in a ratio of 1:20 to 20:1 wherein at least one is hydrophilic; at least one lubricant(s) in an amount less than 6% w/w of the composition; and optionally other pharmaceutically acceptable excipients; wherein the composition provides therapeutic concentrations of active agent(s) for extended periods of time.

It is also an objective of the present invention to provide novel modified release pharmaceutical composition comprising at least one active agent(s) selected from a group comprising HMG CoA reductase inhibitor such as statins and the like, or its pharmaceutically acceptable salts, hydrates, polymorphs, esters, and derivatives.

It is also an objective of the present invention to provide novel modified release pharmaceutical composition comprising at least one active agent(s) selected from a group comprising tacrolimus, oxcarbazepine, levetiracetam, quetiapine, tolterodine, famciclovir, and the like, or its pharmaceutically acceptable salts, hydrates, polymorphs, esters, and derivatives.

It is also an objective of the present invention to provide novel modified release pharmaceutical composition comprising at least one HMG CoA reductase inhibitor, preferably a statin, more preferably atorvastatin or simvastatin, or its pharmaceutically acceptable salts, hydrates, polymorphs, esters, and derivatives thereof as active agent(s); a polymer system in an amount less than about 80% w/w of the composition comprising at least two swellable pH independent polymers preferably in a ratio of 1:20 to 20:1 wherein at least one is hydrophilic; and optionally other pharmaceutically acceptable excipients; wherein the composition provides therapeutic concentrations of active agent(s) for extended periods of time.

It is another objective of the present invention to provide process for the preparation of such novel composition, which comprises of the following steps:

i) mixing the active agent(s) and components of the polymer system,
ii) optionally adding one or more other pharmaceutically acceptable excipients, and
iii) formulating the mixture into a suitable dosage form.

It is yet another objective of the present invention to provide a method of using such composition which comprises administering to a patient in need thereof an effective amount of the composition.

### DETAILED DESCRIPTION OF THE INVENTION

The present invention provides novel modified release pharmaceutical composition comprising at least one active agent(s) or its pharmaceutically acceptable salts, esters, prodrugs, solvates, hydrates, or derivatives thereof, a polymer system in an amount less than about 80% w/w of the composition comprising at least two swellable pH independent polymers wherein at least one is hydrophilic; optionally other pharmaceutically acceptable excipients; wherein the composition provides therapeutic concentrations of active agent(s) for extended periods of time. In an embodiment, preferably the two swellable pH independent polymers are present in a ratio of 1:20 to 20:1. In another embodiment, the polymer system is preferably present in an amount less than about 70% w/w of the composition.

In an embodiment, the compositions additionally comprise at least one diluent(s) in an amount greater than about 2.5% w/w of the composition and/or at least one lubricant(s) in an amount less than about 8% w/w of the composition.

In an embodiment, the present invention provides novel modified release pharmaceutical composition comprising at least one active agent(s) or its pharmaceutically acceptable salts, esters, prodrugs, solvates, hydrates, or derivatives thereof; a polymer system in an amount less than about 80% w/w of the composition comprising at least two swellable pH independent polymers wherein at least one is hydrophilic and additionally, at least one pH dependent hydrophilic release controlling polymer; optionally other pharmaceutically acceptable excipients; wherein the composition provides therapeutic concentrations of active agent(s) for extended periods of time.

The active agent of the present invention may be selected from but not limited to a group comprising active agents such as anti-hypertensives; immunosuppressants; anti-inflammatories; diuretics; antiepileptics; antifungals; cholesterol lowering drugs; hormonals; hypoglycemics; antiviral drugs; nasal decongestants; antimicrobials; anti-arthritics; analgesics; anti-cancer drugs; anti-parasitics; proteins; peptides; CNS stimulants; CNS depressants; 5HT inhibitors; HMG CoA reductase inhibitors; anti-schizophrenics; anti-alzheimer drugs; anti-psoriatics; steroidals; oligonucleotides; anti-ulcer drugs; proton pump inhibitors; anti-asthmatics; immunomodulators; thrombolitics and vitamins, or their pharmaceutically acceptable salts, hydrates, polymorphs, esters, and derivatives; or mixtures thereof. In an embodiment, the active agent(s) of the present invention is selected from but not limited to a group comprising tacrolimus, oxcarbazepine, levetiracetam, quetiapine, tolterodine, famciclovir, and the like, or its pharmaceutically acceptable salts, hydrates, polymorphs, esters, and derivatives.

In a preferred embodiment, the active agent(s) of the present invention is an HMG CoA reductase inhibitor, preferably a statin, or its pharmaceutically acceptable salts, hydrates, polymorphs, esters, and derivatives thereof. The statin is selected from but not limited to a group comprising simvastatin, atorvastatin, pravastatin, lovastatin, cerivastatin, rosuvastatin, and fluvastatin, or pharmaceutically acceptable salts, hydrates, polymorphs, esters, and derivatives thereof, used either alone or in combination thereof. In another preferred embodiment, the active agent of the present invention is an immunomodulator such as tacrolimus or pharmaceutically acceptable salts, hydrates, polymorphs, esters, and derivatives thereof.

The composition is formulated into a suitable dosage form and provides therapeutic concentrations of active agent(s) for extended periods of time. The novel compositions of the present invention release the active agent for a period of about 10-24 hours, preferably from about 15-24 hours. In an embodiment, the said composition provides an initial lag time for the release of the active agent(s) from the dosage form either in the in vitro dissolution study or in vivo upon administration into the body or provides a lag time for absorption of the active agent(s) in vivo. In an embodiment, the said composition provides an initial lag time from wherein only about 5-15% of active agent(s) or less is released, followed by a sustained release of active agent(s), particularly in case of poorly soluble active agents. The release is erosion controlled such that the active agent leaches into the surrounding environment as long as the polymer blend containing the active agent(s) erodes out of the formulation in controlled manner. The polymer system used in the present invention is unique and act to produce the desired release profile of the active agent. In an embodiment, the composition is a modified release preparation with more than two different rate controlling polymers acting synergistically or wherein one polymer potentiates the activity of the other when incorporated together; hence requiring less quantity of polymer as compared to when incorporated alone or without any one polymer. Also the lag time in the release profile of the active agent can be obtained with the polymer system of the present invention without any need for a functional coating or any other mechanism like osmotic pressure etc. Moreover the direct compression technique or compaction granulation technique preferably used to formulate the compositions of the present invention are simple and thus involve low processing cost. In an embodiment, the compositions of the present invention are preferably useful for active agents for which the stomach and/or the upper part of the gastrointestinal tract are the preferred site of absorption. In another embodiment, the compositions of the present invention are formulated as gastroretentive dosage forms, wherein the said dosage form is retained for a prolonged duration in the gastrointestinal tract thus providing a sustained or controlled release of the active agent(s).

In an embodiment of the present invention wherein a statin is used as an active agent, the compositions preferably release the active agent after a lag time of at least about 0.5 hour, preferably after a lag time of about 1 hour, more preferably after a lag time of 2-3 hours. In a further embodiment, such compositions comprising statin as active agent are preferably administered during bedtime and more preferably once-a-day. It is believed that the human body synthesizes high amounts of cholesterol during the hours of sleep and it is thus desirable to provide therapeutic levels of the HMG CoA reductase inhibitors during this time. Hence, in an embodiment, the compositions of the present invention comprising statin as the active agent are so designed that during the initial lag time i.e. during the daytime lesser amount of active agent is released and subsequently a sustained amount of drug is released during the hours of sleep in order to provide the desirable enhanced therapeutic effect of the active agent. In a further embodiment, the compositions of the present invention comprising statin as active agent are particularly effective in inhibiting the biosynthesis of cholesterol in the liver by inhibition of the HMG CoA reductase since these are formulated in such a manner so as to deliver the maximum quantity of the active agent to the liver tissues and minimum quantity to the peripheral tissues so as to minimize any adverse effects associated with the presence of a higher quantity of the active agent in the latter.

In an embodiment of the present invention, the diluent is selected from but not limited to a group comprising lactose, cellulose, microcrystalline cellulose, mannitol, dicalcium phosphate, pregelatinized starch, and the like, used either alone or in combination thereof. Preferably the diluent(s) is present in an amount greater than about 2.5% w/w of the composition, more preferably in an amount greater than about 20% w/w of the composition, most preferably in an amount greater than about 70% w/w of the composition. In another embodiment, the diluent(s) used in the compositions of the present invention can also additionally function as channel forming agents, preferably when used in lower concentrations such as in an amount less than about 50% w/w of the composition.

The composition of the present invention comprises a polymer system in an amount less than about 80% w/w of the composition comprising at least two swellable pH independent polymers wherein at least one is hydrophilic. Optionally the polymer system comprises additionally at least one pH dependent hydrophilic release controlling polymer.

Suitable polymers for use in the compositions of the present invention may be linear, branched, dendrimeric, or star polymers, and include synthetic hydrophilic polymers as well as semi-synthetic and naturally occurring hydrophilic polymers. The polymers may be homopolymers or copolymers, if copolymers, either random copolymer, block copolymers or graft copolymers. Synthetic hydrophilic polymers useful herein include, but are not limited to polyalkylene oxides, particularly poly(ethylene oxide), and poly (ethylene oxide)-poly(propylene oxide) copolymers; acrylic acid and methacrylic acid polymers, copolymers and esters thereof, preferably formed from acrylic acid, methacrylic acid, methyl acrylate, ethyl acrylate, methyl methacrylate, ethyl methacrylate, and copolymers thereof; polysaccharide gums; and the like used alone or mixtures.

In a further embodiment of the present invention, the swellable pH independent polymer of the polymer system is particularly a polyalkylene oxide preferably poly (ethylene oxide), which is a linear polymer of unsubstituted ethylene oxide. Preferred poly(ethylene oxide)s are those available in the Polyox® family of trademarks, e.g., Polyox® 303, Polyox® Coag, Polyox® 301, Polyox® WSR N-60K, Polyox® WSR 1105 and Polyox® WSR N-80 (Union Carbide Chemicals and Plastics Company Inc. of Danbury, Conn., USA) used alone or in combination thereof.

In a further embodiment, the hydrophilic swellable pH independent polymer used in the polymer system of the present invention is selected from but not limited to a group comprising polysaccharide gums which are natural and modified (semi-synthetic) or synthetic or their combinations, including but not limited to xanthan gum, veegum, agar, guar gum, locust bean gum, gum arabic, okra gum, bentonite, arabinoglactin, pectin, tragacanth, scleroglucan, dextran, amylose, amylopectin, dextrin, and the like or mixtures thereof. Preferably xanthan gum is used in the present invention.

In a still further embodiment, at least one additional pH dependent hydrophilic release controlling polymer used in the present invention is selected from a group comprising alginic acid or other alginates such as alginic acid derivatives e.g. sodium alginate HVCR (sodium alginate high viscosity controlled release grade) and propylene glycol alginate, or crosslinked polyacrylic acids preferably those with a viscosity ranging from about 4,000 to about 40,000 centipoises for a 1% aqueous solution at 25° C. Examples include but not limited to Carbopol® 971P, 974P and 934P and 71G; sodium polyacrylate grades such as Water Lock® A180, A220 and the like which are starch/acrylates/acrylamide copolymers. In a further embodiment of the present invention, the polymer system additionally comprises glyceryl behenate such as Compritol® 888.

In a still further embodiment, the compositions of the present invention additionally comprise a solubilizing agent. It is known that the challenge associated particularly with the poorly water soluble active agent(s) is to enhance intrinsic solubility, thereby improving oral bioavailability. At least one solubilizing agent(s), and more preferably a surfactant, optionally along with one or more other solubilizer(s) is additionally used in the present invention. Those surfactants, which are overall hydrophilic in nature, especially ethylene oxide-propylene oxide copolymer surfactants (sometimes referred to as ‘poloxamers’) are preferred. The class of surfactants marketed under the trademark Pluronic® and sold under the trade names Lutrol® and Monolan® are also useful. Of the class of Pluronic® surfactants, Pluronic® F68 are especially preferred. Other solubilizing agents include the polyalkylene glycol and their derivatives, for example, Gelucire® such as Gelucire® 50/13 (Gattefosse), which is a polyethylene glycol-32 glyceryl palmitostearate ester (HLB 13); glyceryl palmitostearate; polyoxyethylene alkyl ethers such as polyoxyethylene stearyl ether, polyoxyethylene oleyl ether and polyoxyethylene cetyl ether which are available under the Brij® and Cetomacrogol® series trade names; polyvinylpyrrolidone (such as PVP K30, PVPK90, Kollidon® VA 64, and the like); polar solvents such as alcohol, acetone, alkylene glycol, polyalkylene glycol and the like; used either alone or in combination thereof. In a preferred embodiment, the polar solvent as described herein is preferably a polyalkylene glycol, including, e.g., polyethylene glycol (PEG) such as those having an average molecular weight ranging from about 1,000 to about 15,000, and more preferably from about 1,500 to about 12,000, e.g. PEG 3350.

In a preferred embodiment of the present invention, the polymer system comprises polyethylene oxide and xanthan gum as the two swellable pH independent polymers and Carbopol® 71G as a pH dependent hydrophilic release controlling polymer. In an embodiment, the ratio of the two swellable pH independent polymers of the polymer system of the present invention is 1:20 to 20:1, preferably 1:10 to 10:1 by weight of the composition.

In the present invention, the lubricant used is selected from but not limited to a group comprising magnesium stearate, calcium stearate, sodium stearate, stearic acid, sodium stearyl fumarate, hydrogenated cotton seed oil (sterotex), talc, and waxes, including but not limited to, beeswax, carnauba wax, cetyl alcohol, glyceryl stearate, glyceryl palmitate, glyceryl behenate, hydrogenated vegetable oils, stearyl alcohol and the like, used alone or in combination thereof.

The pharmaceutically acceptable excipients of the present invention are selected from but not limited to a group comprising diluents, disintegrants, binders, fillers, bulking agents, anti-adherants, anti-oxidants, buffering agents, colorants, flavoring agents, coating agents, plasticizers, organic solvents, stabilizers, preservatives, lubricants, glidants, chelating agents, and the like known to the art used either alone or in combination thereof.

In another embodiment, the modified release dosage form of the present invention is in the controlled release form, sustained release form, timed release form, pulsatile release form, prolonged release form or delayed release form, or in a combination of immediate release form and controlled release form. In an embodiment, the modified release compositions can be formulated with the objective of either releasing the drug in a sustained or controlled manner for an extended period of time or releasing a portion of the drug immediately followed by a sustained or controlled release of drug. It will be appreciated that certain excipients used in the present composition can serve more than one purpose.

In another embodiment of the present invention is provided a process for the preparation of such novel compositions. In an embodiment, the process of preparation comprises of the following steps:
i) mixing the active agent(s) and components of the polymer system,
ii) optionally adding a lubricant(s) and/or one or more other pharmaceutically acceptable excipients, and
iii) formulating the mixture into a suitable dosage form.

In another embodiment of the present invention is provided a process for the preparation of such novel composition which comprises of the following steps:
i) mixing the active agent(s) with solubilizing agent(s) optionally with diluent(s),
ii) melting the material of step (i) to form a liquid mass followed by cooling to obtain a semisolid mass,
iii) adding the components of the polymer system optionally with diluent(s) to the material of step (ii) followed by mixing, iv) adding a lubricant to the material of step (iii) with mixing,
v) optionally adding one or more other pharmaceutically acceptable excipients, and
vi) formulating the mixture into a suitable dosage form.

In an embodiment, the compositions of the present invention are preferably prepared as oral dosage forms, more preferably in the form of compressed tablets, moulded tablets, multilayer tablets such as a bilayer tablets, mini-tablets, capsules, pellets, granules and products prepared by extrusion or film cast technique, and the like. The tablets may be optionally coated with a nonfunctional coating to form a nonfunctional layer. The tablets/minitablets may be optionally filed into capsules. The tablets can be prepared by either direct compression, dry compression (slugging), or by granulation. In a preferred embodiment of the present invention, the oral composition is prepared by direct compression or compaction granulation. The granulation technique is either aqueous or non-aqueous. The non-aqueous solvent used is selected from a group comprising ethanol, isopropyl alcohol or methylene chloride.

In yet another embodiment, the novel controlled release pharmaceutical compositions of the present invention is intended to reduce the adverse effects or side effects of the active agent(s) by controlling the peak plasma concentration (Cₘₐₓ) such that the concentration of the active agent(s) are substantially below the toxic levels at any point of time. Also the steady state concentrations of the active agent(s) do not exhibit substantial fluctuations. The reduced incidence of side effects is thus intended to improve patient compliance with the therapy.

In a still further embodiment of the present invention is provided a method of using such novel modified release compositions. The method of treatment or use of the compositions of the present invention comprises administering to a patient in need thereof an effective amount of the composition. The compositions of the present invention are useful for the treatment of specific diseases or disorders for which the specific active agent used in making the composition is indicated as known to the art. For example, the compositions comprising statin as the active agent are useful for lowering cholesterol levels and for the treatment of hyperlipidemia. In an embodiment the composition of the present invention comprising tacrolimus is useful particularly for the prophylaxis of organ rejection in patients receiving allogeneic liver or kidney transplants or any other immunomodulator indicated disorder(s).

### EXAMPLES

### Example 1

| S. No. | Ingredient | Quantity (% w/w) |
|---|---|---|
| i) | Simvastatin | 15.53 |
| ii) | Lactose anhydrous | 62.97 |
| iii) | Xanthan gum | 4.00 |
| iv) | Polyethylene oxide | 12.00 |
| v) | Crosslinked polyacrylic acids (Carbopol ® | 4.00 |
| | 71G) | |
| vi) | Magnesium stearate | 1.50 |

### Procedure:

1) Sift Simvastatin and Lactose anhydrous through #40 sieve.
2) Mix the material of step (1) in polygonal blender to get a uniform mixture.
3) Sift Xanthan gum through #60 sieve and mix with the material of step (2).
4) Lubricate the step (3) granules with #60 sieve passed magnesium stearate.
5) Compact the step (4) granules and pass the compact through #30 sieve.
6) Sift Polyethylene oxide and Carbopol® 71G through #30 sieve.
7) Mix step (6) granules with the step (5) granules in a polygonal blender.
8) Lubricate the step (7) granules with #60 sieve passed Magnesium stearate.
9) Compress the material of step (8) into tablets using a tablet compression machine.

### Example 2

| | | Quantity |
|---|---|---|
| S. No. | Ingredient | (% w/w) |
| i) | Tacrolimus | 1.06 |
| ii) | PEG 32 Glyceryl stearate (Gelucire ® 44/14) | 5.00 |
| iii) | Lactose anhydrous | 82.44 |
| iv) | Locust bean gum | 3.00 |
| v) | Polyethylene oxide | 4.50 |
| vi) | Crosslinked polyacrylic acids (Carbopol-71G) | 3.00 |
| vii) | Magnesium stearate | 1.00 |

### Procedure:

1) Melt the specified quantity of PEG 32 Glyceryl stearate at about 60° C.
2) Sift Tacrolimus through #60 sieve and add to the material of step (1).
3) Allow the Tacrolimus to dissolve in melted PEG 32 Glyceryl stearate.
4) Sift partial quantity (about 60%) of Lactose anhydrous through #40 sieve and mix with the material of step (3).
5) Mix the material of step (4) and cool to obtain a uniform solid free flowing blend.
6) Divide the blend of step (5) into two parts in 1:3 ratio and add Lactose anhydrous (about 20%) to the smaller part and compress into immediate release (IR) minitablets.
7) Add remaining quantity of Lactose anhydrous (about 20%) and Locust bean gum (passed through #60 sieve) to the larger part of step (6) blend and mix.
8) Sift Polyethylene oxide and Carbopol-71G through #30 sieve and mix with the step (7) granules.
9) Lubricate the step (8) granules with #60 sieve passed Magnesium stearate and compress into sustained release (SR) minitablets.
10) Fill the minitablets (one IR and three SR minitablets) into hard gelatin capsule.

### Example 3

| S. No. | Ingredient | Quantity (% w/w) |
|---|---|---|
| i) | Simvastatin | 13.5 |
| ii) | Mannitol | 65.0 |
| iii) | Amylose | 5.0 |
| iv) | Polyethylene oxide | 15.0 |
| v) | Magnesium stearate | 1.5 |

### Procedure:

1) Sift Simvastatin and Mannitol through #40 sieve.
2) Mix the material of step (1) to get a uniform mixture.
3) Sift Amylose through #60 sieve and mix with the material of step (2).
4) Lubricate the step (3) granules with #60 sieve passed magnesium stearate.
5) Compact the step (4) granules and pass the compact through #30 sieve completely.
6) Sift Polyethylene oxide through #30 sieve.
7) Mix step (6) granules with the step (5) granules in a polygonal blender.
8) Lubricate the step (7) granules with #60 sieve passed Magnesium stearate.
9) Compress the step (8) granulation into tablets using a tablet compression machine.

### Example 4

| S. No. | Ingredient | Quantity (% w/w) |
|---|---|---|
| i) | Fluvastatin Sodium | 28.14 |
| ii) | Lactose anhydrous | 42.36 |
| iii) | Polyethylene oxide | 20.00 |
| iv) | Xanthan gum | 1.50 |
| v) | Crosslinked polyacrylic acids | 6.00 |
| vi) | Purified water | q.s. (lost in processing) |
| vii) | Magnesium stearate | 2.00 |

### Procedure:

1) Sift Fluvastatin Sodium and Lactose anhydrous through #40 sieve.
2) Mix the material of step (1) in polygonal blender to get a uniform mixture.
3) Sift Polyethylene oxide, Xanthan gum and Crosslinked polyacrylic acids through #30 sieve and mix with the material of step (2).
4) Granulate the material of step (3) with Purified water.
5) Sift the material of step (4) through #24 and dry the granules.
6) Sift the dried granules through #30 sieve.
7) Lubricate the material of step (6) with #60 sieve passed Magnesium stearate.
8) Compress the step (7) granulation into tablets using tablet compression machine.

### Example 5

| S. No. | Ingredient | Quantity (% w/w) |
|---|---|---|
| i) | Pravastatin sodium | 10.0 |
| ii) | Microcrystalline cellulose | 67.0 |
| iii) | Locust bean gum | 10.0 |
| iv) | Poly(ethylene oxide)- | 12.0 |
| | poly(propylene oxide) copolymer | |
| v) | Sodium stearyl fumarate | 1.0 |

### Procedure:

1) Sift Pravastatin sodium and Microcrystalline cellulose through #40 sieve.
2) Mix the material of step (1) to get a uniform mixture.
3) Sift Locust bean gum through #60 sieve and mix with the material of step (2).
4) Sift Poly(ethylene oxide)-poly(propylene oxide) copolymer through #30 sieve.
5) Mix the material of step (3) with the material of step (4).
6) Lubricate the step (5) material with #60 sieve passed magnesium stearate.
7) Fill the material of step 6 into a hard gelatin capsule.

### Example 6

| S. No. | Ingredient | Quantity (% w/w) |
|---|---|---|
| i) | Lovastatin | 10.0 |
| ii) | Maltodextrin | 61.5 |
| iii) | Sodium alginate HVCR | 3.0 |
| iv) | Amylopectin | 7.0 |
| v) | Polyethylene oxide | 15.0 |
| vi) | Poloxamer | 2.0 |
| vii) | Purified water | q.s. (lost in processing) |
| viii) | Stearic acid | 1.5 |

### Procedure:

1) Sift Lovastatin and Maltodextrin through #40 sieve.
2) Mix the material of step (1) to obtain a uniform mixture.
3) Sift Sodium alginate HVCR and Amylopectin through #60 sieve and mix them with the material of step (2).
4) Sift Polyethylene oxide through #30 sieve.
5) Mix the material of step (3) with the material of step (4).
6) Dissolve Poloxamer in Purified water and granulate the material of step (5) with the solution thus obtained.
7) Dry the step (6) granules and pass the material through #30 sieve.
8) Lubricate the step (7) granules with #60 sieve passed Stearic acid.
9) Compress the step (8) granulation into tablets using tablet compression machine.

### Example 7

| | | Quantity |
|---|---|---|
| S. No. | Ingredient | (% w/w) |
| i) | Atorvastatin calcium | 15.0 |
| ii) | Lactose | 43.5 |
| iii) | Microcrystalline cellulose | 15.0 |
| iv) | Xanthan gum | 5.0 |
| v) | Polyethylene oxide | 15.0 |
| vi) | Crosslinked polyacrylic acids (Carbopol ® 971P) | 5.0 |
| vii) | Glyceryl stearate | 1.5 |

### Procedure:

1) Sift Atorvastatin, Lactose and Microcrystalline cellulose through #40 sieve.
2) Mix the material of step (1) to get a uniform mixture.
3) Sift Xanthan gum through #60 sieve and mix with the material of step (2).
4) Sift Polyethylene oxide and Crosslinked polyacrylic acids through #30 sieve.
5) Mix step (3) material with the step (4) material in a polygonal blender.
6) Lubricate the step (5) material with #60 sieve passed Glyceryl stearate.
7) Compress the step (6) material into tablets using tablet compression machine.

### Example 8

| | | Quantity |
|---|---|---|
| S. No. | Ingredient | (% w/w) |
| i) | Tacrolimus | 1.06 |
| ii) | Polyethylene glycol-32 glyceryl palmitostearate | 5.00 |
| | (Gelucire ® 50/13) | |
| iii) | Dicalcium phosphate anhydrous | 8.94 |
| iv) | Xanthan gum | 5.00 |
| v) | Lactose anhydrous | 59.00 |
| vi) | Polyethylene oxide | 15.00 |
| vii) | Crosslinked polyacrylic acids | 5.00 |
| viii) | Magnesium stearate | 1.00 |

### Procedure:

1) Sift Tacrolimus through #40 sieve and mix with Polyethylene glycol-32 glyceryl palmitostearate.
2) Melt the material of step (1) to form a liquid mass.
3) Sift Dicalcium phosphate anhydrous through #40 sieve and mix with the material of step (2) to form a semisolid mass.
4) Sift Xanthan gum through #60 sieve and mix with the material of step (3).
5) Sift Lactose anhydrous through #60 sieve and mix with the material of step (4).
6) Sift Polyethylene oxide and Crosslinked polyacrylic acids through #30 sieve and mix with the material of step (5).
7) Lubricate the material of step (6) with #60 passed Magnesium stearate.
8) Compress the granulation of step (7) to obtain the tablets.

### Example 9

| S. No. | Ingredient | Quantity (% w/w) |
|---|---|---|
| i) | Rosuvastatin | 10.0 |
| ii) | Glyceryl behenate (Compritol ® 888) | 15.0 |
| iii) | Xanthan gum | 5.0 |
| iv) | Lactose anhydrous | 49.0 |
| v) | Polyethylene oxide | 15.0 |
| vi) | Crosslinked polyacrylic acids | 5.0 |
| vii) | Magnesium stearate | 1.0 |

### Procedure:

1) Sift Rosuvastatin through #40 sieve and mix with Glyceryl behenate.
2) Melt the material of step (1) to form a liquid mass.
3) Cool the material of step (3) to form a semisolid mass.
4) Sift Xanthan gum through #60 sieve and mix with the material of step (3).
5) Sift Lactose anhydrous through #60 sieve and mix with the material of step (4).
6) Sift Polyethylene oxide and Crosslinked polyacrylic acids through #30 sieve and mix with the material of step (5).
7) Lubricate the material of step (6) with #60 passed Magnesium stearate.
8) Fill the granulation of step (7) into hard gelatin capsule.

### Example 10

| | | Quantity |
|---|---|---|
| S. No. | Ingredient | (% w/w) |
| i) | Levetiracetam | 62.8 |
| ii) | Microcrystalline cellulose | 6.2 |
| iii) | Xanthan gum | 5.0 |
| iv) | Hydroxypropyl methylcellulose (HPMC ® K100M) | 5.0 |
| v) | Polyethylene oxide | 15.0 |
| vi) | Crosslinked polyacrylic acids (Carbopol ® 971P) | 5.0 |
| vii) | Magnesium stearate | 1.0 |

### Procedure:

1) Sift Levetiracetam, Microcrystalline cellulose, Xanthan gum and Crosslinked polyacrylic acids through #40 sieve.
2) Mix the material of step (1) to get a uniform blend.
3) Compact the step (2) blend and pass the compact through #22 sieve.
4) Sift Polyethylene oxide through #30 sieve and mix with the material of step (3).
5) Sift Hydroxypropyl methylcellulose through #60 sieve and mix with the material of step (4).
6) Lubricate the step (5) granules with #60 sieve passed Magnesium stearate.
7) Compress the step (6) granulation into tablets using tablet compression machine.

### Example 11

| S. No. | Ingredient | Quantity (% w/w) |
|---|---|---|
| i) | Famciclovir | 68.63 |
| ii) | Lactose | 10.37 |
| iii) | Xanthan gum | 5.00 |
| iv) | Polyethylene oxide (PEO ® 301) | 10.00 |
| v) | Crosslinked polyacrylic acids | 5.00 |
| vi) | Glyceryl behenate | 1.00 |

### Procedure:

1) Sift Famciclovir, Lactose and Crosslinked polyacrylic acids through #40 sieve.
2) Mix the material of step (1) to get a uniform mixture.
3) Compact the step (2) granules.
4) Pass the compact through #20 sieve completely.
5) Sift Polyethylene oxide through #30 sieve and mix with the material of step (4).
6) Sift Xanthan gum through #60 sieve and mix with step (5) granules.
57) Lubricate the step (6) granules with #60 sieve passed Glyceryl behenate.
8) Compress the step (7) granulation into tablets using tablet compression machine.

### Example 12

| | | Quantity |
|---|---|---|
| S. No. | Ingredient | (% w/w) |
| i) | Quetiapine Fumarate | 38.44 |
| ii) | Lactose monohydrate | 40.06 |
| iii) | Xanthan gum | 3.00 |
| iv) | Polyethylene oxide | 8.00 |
| v) | Crosslinked polyacrylic acids (Carbopol ® 71G) | 3.00 |
| vi) | Hydroxypropyl methylcellulose (HPMC ® E5) | 6.00 |
| vii) | Sodium stearyl fumarate | 1.50 |

### Procedure:

1) Sift Quetiapine Fumarate and Lactose monohydrate through #40 sieve.
2) Mix the material of step (1) to get a uniform mixture.
3) Sift Xanthan gum through #60 sieve and mix with the material of step (2).
4) Sift Crosslinked polyacrylic acids through #30 sieve and mix with the material of step (3).
5) Lubricate the step (4) granules with #60 sieve passed Sodium stearyl fumarate.
6) Compact the step (5) granules.
7) Pass the compact through #20 sieve completely.
8) Sift Polyethylene oxide through #30 sieve and mix with the material of step (7).
9) Sift Hydroxypropyl methylcellulose through #40 sieve and mix with step (8) granules.
10) Lubricate the step (9) granules with #60 sieve passed Sodium stearyl fumarate.
11) Compress the step (10) granulation into tablets using tablet compression machine.

### Example 13

| S. No. | Ingredient | Quantity (% w/w) |
|---|---|---|
| i) | Tolterodine tartrate | 1.35 |
| ii) | Glyceryl Palmitostearate (Precirol ® ATO5) | 15.00 |
| iii) | Dicalcium Phosphate Anhydrous | 8.65 |
| iv) | Polyethylene oxide | 12.00 |
| v) | Crosslinked polyacrylic acids | 4.00 |
| vi) | Xanthan gum | 2.00 |
| vii) | Silicified microcrystalline cellulose | 56.00 |
| viii) | Sodium stearyl fumarate | 1.00 |

### Procedure:

1) Melt Glyceryl Palmitostearate at a temperature of about 55-60° C.
2) Sift Tolterodine tartrate through #40 sieve and add to the material of step (1) and mix to form uniform dispersion.
3) Sift Dicalcium phosphate anhydrous through #40 sieve and mix with the material of step (2) to get dry granules.
4) Sift Silicified microcrystalline cellulose through #40 sieve and mix with the material of step (3).
5) Pass the step (4) material through #40 sieve.
6) Sift Xanthan gum through #40 sieve and mix with the material of step (5).
7) Sift Polyethylene oxide through #30 sieve and mix with the material of step (6).
8) Sift Crosslinked polyacrylic acids through #30 sieve and mix with the material of step (7).
9) Sift Sodium stearyl fumarate through #40 and mix with material of step (8).
10) Compress the step (9) granules to form a minitablets.
11) Fill the minitablets in suitable size hard gelatin capsule.

### Example 14

A) Composition for Immediate Release (IR) layer:

| S. No. | Ingredient | Quantity (% w/w) |
|---|---|---|
| i) | Oxcarbazepine | 60.25 |
| ii) | Anhydrous lactose | 16.75 |
| iii) | Hydroxyethyl cellulose | 2.00 |
| iv) | Crospovidone | 5.00 |
| v) | Microcrystalline cellulose | 15.00 |
| vi) | Magnesium stearate | 1.00 |

### Procedure:

1) Sift Oxcarbazepine and Lactose anhydrous through #40 sieve.
2) Mix the material of step (1) in polygonal blender for 10 min.
3) Sift Hydroxyethyl cellulose through #40 sieve and mix with the material of step (2).
4) Sift half the quantity of Magnesium stearate through #60 sieve and mix with the material of step (3).
5) Compact the material of step (4) in a roller compactor.
6) Pass the compact through #20 sieve completely.
7) Sift Crospovidone through #40 sieve and mix with material of step (6).
8) Sift Microcrystalline cellulose through #40 sieve and mix with step (7) granules.
9) Sift remaining quantity of Magnesium stearate through #60 sieve and mix with the material of step (8).

B) Composition for Sustained Release (SR) layer:

| | | Quantity |
|---|---|---|
| S. No. | Ingredient | (% w/w) |
| i) | Oxcarbazepine | 70.29 |
| ii) | Anhydrous lactose | 12.71 |
| iii) | Xanthan Gum | 4.00 |
| iv) | Polyethylene oxide | 8.00 |
| v) | Crosslinked polyacrylic acids (Carbopol ® 71G) | 4.00 |
| vi) | Magnesium stearate | 1.00 |

### Procedure:

1) Sift Oxcarbazepine and Lactose anhydrous through #40 sieve.
2) Mix the material of step (1) in polygonal blender.
3) Sift Xanthan gum through #60 sieve and mix with the material of step (2).
4) Sift half the quantity of Magnesium stearate through #60 sieve and mix with the material of step (3).
5) Compact the step (4) blend in a roller compactor.
6) Pass the compact through #20 sieve completely.
7) Sift Crosslinked polyacrylic acids through #30 sieve and mix with the material of step (6).
8) Sift Polyethylene oxide through #30 sieve and mix with the material of step (7).
9) Lubricate the step (8) granules with remaining quantity of #60 sieve passed Magnesium stearate.

### C) Preparation of Bilayer Tablet:

Bilayer tablets was manufactured on a Rotary Multi-layer Tablet Press by filling the die step-wise with the contents of two layers (Step-9 granules of A and B) with subsequent compression into tablets. After the die is filled with the content of one layer, the tableting punches compress the powder bed slightly before the die is additionally filled with the content of the succeeding layer and final compression leading to bilayer tablet.

1-25. (canceled)

26. A modified release pharmaceutical composition comprising at least one active agent(s) or pharmaceutically acceptable salts, esters, prodrugs, solvates, hydrates, or derivatives thereof; a polymer system in an amount less than about 80% w/w of the composition consisting of two pH independent swellable polymers which are polyalkylene oxide and a hydrophilic polymer in a ratio of 1:20 to 20:1; optionally other pharmaceutically acceptable excipients; wherein the said composition provides an initial lag time wherein 5-15% of active agent(s) or less is released followed by a sustained release of active agent(s), such that the composition provides therapeutic concentrations of active agent(s) for extended periods of time.

27. The composition as claimed in claim 26, wherein the active agent is selected from a group comprising HMG CoA reductase inhibitors, immunomodulators, and pharmaceutically acceptable salts, hydrates, polymorphs, esters, and derivatives thereof.

28. The composition as claimed in claim 26, wherein the active agent is selected from a group comprising statin, tacrolimus, oxcarbazepine, levetiracetam, quetiapine, tolterodine, famciclovir, and pharmaceutically acceptable salts, hydrates, polymorphs, esters, and derivatives thereof.

29. The composition as claimed in claim 26, wherein the excipient is a diluent selected from a group comprising lactose, cellulose, microcrystalline cellulose, mannitol, dicalcium phosphate, and pregelatinized starch, used either alone or in combination thereof.

30. The composition as claimed in claim 26, wherein the hydrophilic polymer is selected from a group comprising polysaccharide gums, alginic acid, alginic acid derivatives, arabinoglactin, pectin, tragacanth, scleroglucan, dextran, amylose, amylopectin, and dextrin.

31. The composition as claimed in claim 30, wherein the polysaccharide gum is selected from a group comprising xanthan gum, veegum, agar, guar gum, locust bean gum, gum arabic, and okra gum.

32. The composition as claimed in claim 26, wherein the polymer system comprises an additional pH dependent hydrophilic polymer.

33. The composition as claimed in claim 32, wherein the pH dependent hydrophilic polymer is selected from a group comprising crosslinked polyacrylic acids and polyacrylates, alginic acid, or a derivative thereof.

34. The composition as claimed in claim 26, wherein the polymer system consists of polyethylene oxide and xanthan gum as the two pH independent swellable polymers; and a crosslinked polyacrylic acid or polyacrylate as a pH dependent hydrophilic polymer.

35. The composition as claimed in claim 34, wherein the polymer system consists of xanthan gum, polyethylene oxide and a crosslinked polyacrylic acid or polyacrylate in a ratio of 1:1.5:1 to 1:13.3:4.

36. The composition as claimed in claim 26, wherein the composition additionally comprises a solubilizing agent.

37. The composition as claimed in claim 36, wherein the solubilizing agent is selected from a group comprising ethylene oxide-propylene oxide copolymer surfactants, polyalkylene glycol and their derivatives, polyoxyethylene alkyl ethers, polyvinylpyrrolidone, and polar solvents, used either alone or in a combination thereof.

38. The composition as claimed in claim 26, wherein the composition additionally comprises a lubricant in an amount less than 8% w/w of the composition.

39. The composition as claimed in claim 38, wherein the lubricant is selected from a group comprising magnesium stearate, calcium stearate, sodium stearate, stearic acid, sodium stearyl fumarate, hydrogenated cotton seed oil, talc, and waxes used alone or in combination thereof.

40. The composition as claimed in claim 26, wherein the pharmaceutically acceptable excipients are selected from a group comprising diluents, disintegrants, binders, fillers, bulking agents, anti-adherents, anti-oxidants, buffering agents, colorants, flavoring agents, coating agents, plasticizers, organic solvents, stabilizers, preservatives, lubricants, glidants, and chelating agents used either alone or a combination thereof.

41. A process of preparation of the composition as claimed in claim 26, which comprises the steps of:
i) mixing the active agent(s) and components of the polymer system,
ii) optionally adding a lubricant(s) and/or one or more other pharmaceutically acceptable excipients, and
iii) formulating the mixture into a suitable dosage form.

42. A process for the preparation of the composition as claimed in claim 26, which comprises the steps of
i) mixing the active agent(s) with solubilizing agent(s) optionally with diluent(s),
ii) melting the material of step (i) to form a liquid mass followed by cooling to obtain a semisolid mass,
iii) adding the components of the polymer system optionally with diluent(s) to the material of step (ii) followed by mixing,
iv) adding a lubricant to the material of step (iii) with mixing,
v) optionally adding one or more other pharmaceutically acceptable excipients, and
vi) formulating the mixture into a suitable dosage form.

43. A method of using a composition as claimed in claim 26, which comprises administering to a patient in need thereof an effective amount of the composition.
